(19) United States
(12) Patent Application Publication
(10) Pub. No.: US 2026/0315215 A1
(43) Pub. Date: Oct. 15, 2026

(54) OSSICULAR PROSTHESIS

(71) Applicant: MED-EL Elektromedizinische Geräte GmbH, Innsbruck (AT)

(72) Inventors: Uwe Steinhardt, Hirrlingen (DE); Dirk Beutner, Göttingen (DE); Clemens Kuen, Imst (AT); Magdalena Schweissgut, Absam (AT)

(21) Appl. No.: 19/475,673

(22) PCT Filed: Apr. 12, 2024
(86) PCT No.: PCT/EP2024/059964

§ 371 Date: Oct. 15, 2025

(30) Foreign Application Priority Data
Apr. 21, 2023 (DE) 102023110157.9

(51) Int. Cl. A61F 2/18; A61F 11/00

(52) U.S. Cl. CPC A61F 2/18; A61F 11/00; A61F 2002/183; A61F 2230/0006

(57) ABSTRACT

According to the invention, a partial-ring-shaped coupling element (2), which extends over approximately 270° in a circumferential direction, for an ossicular prosthesis (1) is provided with two supports (7), for example punched-disc type supports, which protrude laterally relative to each other, said supports supporting the ossicular prosthesis (1) on an ossicle so as to prevent tilting, thereby facilitating implantation of the ossicular prosthesis (1).

The invention relates to an auditory ossicular prosthesis having the features of the preamble of claim 1.

Ossicular prostheses serve as a replacement for one or more auditory ossicles in the human middle ear and transmit vibrations of the eardrum generated by sound waves to an existing auditory ossicle, usually the stapes, instead of the replaced auditory ossicle(s). It is also possible for a vibration from an artificial vibration generator implanted in a human ear to be transmitted to an auditory ossicle.

European patent application EP 2 926 771 A1 discloses an ossicular prosthesis with a strip-shaped coupling element which is bent into a partial ring extending over approximately 270° in a circumferential direction such that an opening remains, by which the coupling element can be placed on a rod-shaped part of an auditory ossicle in order to attach the auditory ossicle prosthesis to the auditory ossicle.

One problem with implanting an ossicular prosthesis is that the ossicular prosthesis can tilt when the coupling element is placed on an ossicle and when the coupling element is aligned with the ossicle. As a result, if, for example, a prosthetic body of the ossicular prosthesis rests against the footplate in a hole that a surgeon has drilled through a footplate of a stapes to implant the ossicular prosthesis, instead of being led through the footplate into an inner ear with an annular gap between the prosthetic body and the hole in the footplate of the stapes, sound transmission into the inner ear is impaired. The prosthesis body is a kind of shaft that protrudes outwards from the partially ring-shaped coupling element of the ossicular prosthesis. To make matters worse, a surgeon's view of the middle ear is limited when operating, and therefore a poor fit of the coupling element of the ossicular prosthesis on the ossicle is not always apparent.

The object of the invention is to propose an ossicular prosthesis of the above type, the hold of which is improved against tipping on an ossicle.

This object is achieved according to the invention by the features of claim 1. The auditory ossicle prosthesis according to the invention has a partially ring-shaped coupling element for attaching the auditory ossicle prosthesis to an auditory ossicle. In a circumferential direction, the coupling element extends over more than 180° and less than 360° and preferably over approximately 270° such that the coupling element has an opening through which it can be placed on an auditory ossicle. To be placed on the auditory ossicle, the coupling element is elastically and/or plastically expanded so that the auditory ossicle fits through the opening of the coupling element. After being placed on the auditory ossicle, the coupling element springs back together or is plastically bent back together. Because the coupling element extends over more than 180° in the circumferential direction, it holds on to the auditory ossicle by form-fitting in the radial direction. The coupling element is placed on a rod-shaped part of the auditory ossicle, for example on a bracket of a stirrup, a process or limb of an incus, or a handle of a hammer. The words “stapes,” “anvil,” and “hammer” refer to the auditory ossicles in a human ear. “Process” and “leg” are two terms for the same part of the incus.

The coupling element of the auditory ossicular prosthesis according to the invention has one or more supports which project laterally from the coupling element, wherein “laterally” means projecting from the end faces of the coupling element or perpendicular to a radial and perpendicular to a tangent of the coupling element. The support or supports extend a support on the part of the auditory ossicle onto which the coupling element is placed, in a longitudinal direction of the part of the auditory ossicle and thereby increases a tilt resistance of the coupling element on the auditory ossicle and thus of the auditory ossicle prosthesis as a whole against tilting of the coupling element and the auditory ossicle prosthesis about a transverse axis of the part of the auditory ossicle onto which the coupling element is placed. The invention simplifies implantation of the ossicular prosthesis in a middle ear and increases the probability of a good fit of the coupling element on the ossicle in order to ensure good sound transmission into an inner ear.

The dependent claims relate to advantageous embodiments and further developments of the invention.

Preferably, the coupling element of the auditory ossicular prosthesis according to the invention has a cylindrical inner peripheral surface. The coupling element rests against the auditory ossicle with its inner peripheral surface when the coupling element is placed on the auditory ossicle as intended in such a way that the coupling element encloses the auditory ossicle. In the axial direction of the imaginary cylinder enclosed by the coupling element, the coupling element is preferably short, i.e. the coupling element is preferably narrow in the axial direction in relation to its diameter. The inner circumferential surface of the coupling element is in particular circular-cylindrical, but in embodiments of the invention can also have the shape of a cylinder with a cross-sectional area other than a circle, preferably a round one. An inner surface of the support with which the support rests against the auditory ossicle when the coupling element is placed on the auditory ossicle and is located laterally next to the coupling element on the inner circumferential surface of the coupling element, i.e. on a circumferential surface of the imaginary cylinder that the coupling element encloses. The inner surface of the support is the surface of the support that faces an axis of the imaginary cylinder.

It is also possible to have a conical inner circumferential surface of the coupling element to adapt to, for example, an anvil leg that tapers in one direction. In this case, the inner surface of the support is located on the cone that the coupling element encloses. However, a disadvantage of a conical inner peripheral surface of the coupling element is that the coupling element must be placed on the auditory ossicle with the correct orientation, i.e. the inner peripheral surface of the coupling element must taper in the same direction as the enclosed auditory ossicle. As with a cylindrical inner peripheral surface, a frustoconical inner peripheral surface can have a circular or a round, but not circular, cross-sectional area.

Preferably, the coupling element of the auditory ossicular prosthesis according to the invention has supports on both end faces or on both end edges, which protrude laterally from the coupling element in opposite directions. In particular, two supports are arranged opposite each other on the two end faces of the coupling element and protrude laterally from the coupling element in opposite directions. Supports on both end faces of the coupling element extend the support of the coupling element on the auditory ossicle in the axial direction of the coupling element or in a longitudinal direction of the auditory ossicle on which the auditory ossicle is or will be placed, and in this way improve the support of the coupling element on the auditory ossicle against tipping.

The coupling element may have one support or several supports on one or both end faces, which are arranged evenly or unevenly distributed over a circumference of the coupling element. For example, the coupling element has two supports on one end face or two supports on each end face, which are arranged opposite each other, i.e. the supports are 180° in the circumferential direction offset from each other. The supports thus support the coupling element on the auditory ossicle against tilting in two opposite directions in one tilting plane. This embodiment of the invention is particularly advantageous when there is a main tilt direction, i.e. when it is known in which direction the ossicular prosthesis is subjected to the greatest load. Non-opposite supports are also possible, i.e. an offset of the supports other than 180° in the circumferential direction of the coupling element. Supports can be provided in pairs opposite each other on both end faces of the coupling element and/or supports can be arranged offset from each other on the two end faces in the circumferential direction of the coupling element.

Preferably, the coupling element of the auditory ossicular prosthesis according to the invention has one or two supports on one or both end faces. Versions of the invention with more than two, for example up to five supports on one or both end faces are also possible.

One embodiment of the invention provides a wave-shaped or toothed front edge of the coupling element, wherein the waves or teeth on the front edge of the coupling element form the supports. This embodiment of the invention preferably has more than five supports with short distances in the circumferential direction of the coupling element. “Toothed” means a front edge in the manner of a saw blade or a rack, whereby the teeth, i.e. the supports, can have basically any shape when viewed radially to the coupling element. For example, the teeth or supports are triangular, sawtooth-shaped, rectangular or trapezoidal.

In the circumferential direction, the support is preferably short in relation to the coupling element, i.e. the support extends, for example, only over approximately half a diameter of the coupling element or less in the circumferential direction of the coupling element. As a result, the support increases the flexural rigidity of the coupling element in the radial direction, i.e. in terms of a change in the diameter of the coupling element, it only slightly increases the flexural rigidity and does not make it more difficult to place the coupling element on the auditory ossicle and to press the coupling element firmly onto the auditory ossicle.

Preferably, the coupling element has a partially annular strip which is bent perpendicular to its thickness to the partial ring, i.e. the thickness of the coupling element is radial to the partially annular coupling element. In the circumferential direction outside the support(s) or between the supports, the coupling element in embodiments of the invention has a constant width, wherein the width is a dimension of the coupling element in the axially parallel direction of the partially annular coupling element. For example, the coupling element has a rectangular or trapezoidal or possibly an oval ring cross-section, the larger dimension of which extends axially parallel and the smaller dimension of which extends radially to the partially annular coupling element. The support(s) protrude axially parallel or at a conical angle from one end edge or from both end edges of the partially annular strip. In particular, a cross-section of the strip outside or in the circumferential direction of the coupling element between the supports is constant over a circumference of the coupling element.

The coupling element can be located in a radial plane to an axis of the partially annular coupling element. One embodiment of the invention provides a helical coupling element. This means that the coupling element does not run in a radial plane to its axis, but has a-especially small-gradient, which does not have to be constant.

The support(s) of embodiments of the invention have a hole extending radially through. For example, the support has/has the shape of a circular or non-circular or a polygonal, for example triangular, perforated disc. The hole in the support(s) extends their edge lengths and thus improves the hold of the coupling element or its support(s) on the auditory ossicle through adhesion.

One embodiment of the invention provides a prosthetic body protruding outwards from the coupling element for sound transmission, for example, into the inner ear. The prosthesis body is, for example, a shaft that protrudes radially outwards from the coupling element. In embodiments of the invention, the support of the coupling element is arranged on the coupling element as an extension of the prosthesis body, i.e. in a plane with the prosthesis body. Viewed in a longitudinal direction of the prosthetic body The support is thus located next to the prosthesis body and supports the auditory ossicle prosthesis according to the invention laterally next to the coupling element against tilting about a radial axis of the coupling element or about a transverse axis of the prosthesis body on the auditory ossicle.

In one embodiment of the invention, the coupling element has elongated holes or slots arranged obliquely to the circumferential direction. The elongated holes make it easier to bend the coupling element for a given width of the coupling element. This makes it easier for the coupling element to adapt to the auditory ossicle and causes less bounce.

All features mentioned in the description and/or shown in the drawings can be implemented individually or in any combination in embodiments of the invention. Embodiments of the invention which do not have all, but only some, of the features of a claim, including the independent claim, are possible.

The invention is explained in more detail below with reference to embodiments shown in the drawing. They show:

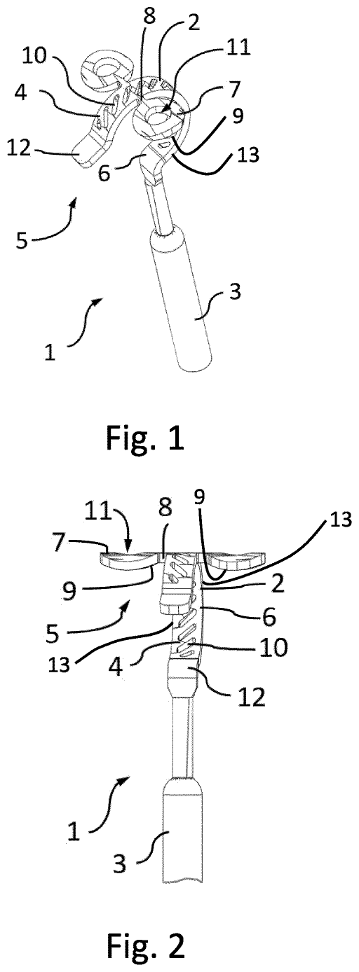

FIG. 1 shows an ossicular prosthesis according to the invention in perspective view;

FIG. 2 is a view of the ossicular prosthesis of FIG. 1;

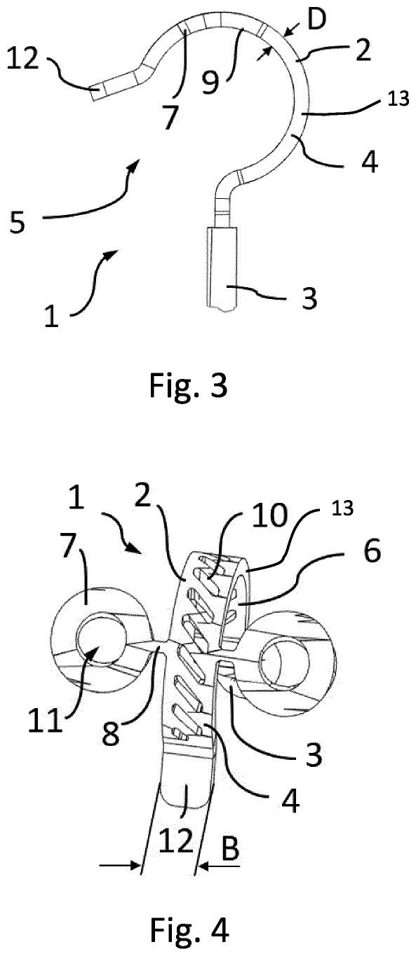

FIG. 3 is a side view of the auditory ossicular prosthesis from FIG. 1, rotated by 90° compared to FIG. 2;

FIG. 4 is a front view of the auditory ossicular prosthesis of FIGS. 1 to 3; and

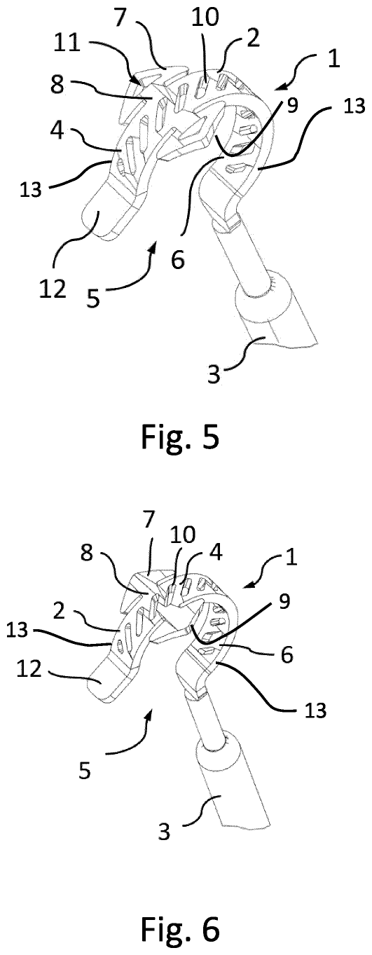

FIGS. 5 to 10 show modified versions of the auditory ossicle prosthesis from FIGS. 1 to 4 according to the invention in a perspective view corresponding to FIG. 1.

In principle, identical parts in the figures are provided with identical reference numbers.

The auditory ossicle prosthesis 1 according to the invention shown in FIGS. 1 to 4 is Intended for replacing one or more auditory ossicles in a human ear. It comprises a coupling element 2, which is intended to be placed on an auditory ossicle, and a prosthetic body 3, which serves to transmit sound from the auditory ossicle to an oval window or into an inner ear in the human ear.

The coupling element 2 is partially ring-shaped, it has a strip 4 which is bent into a partial ring which-in the exemplary embodiment-extends over approximately 270° in a circumferential direction, whereby an opening 5 is formed through which the coupling element 2 can be snapped or placed onto the auditory ossicle. Ends 12 of the strip 4 are bent obliquely or radially outwards, whereby the opening 5 of the coupling element 2 widens outwards in a funnel shape radially to the coupling element 2, i.e. viewed in the axial direction of the coupling element 2 (FIG. 3).

In the exemplary embodiment, the strip 4 has a flat, rectangular cross-section which is wider than it is thick and whose width B extends in an axial direction of the partial ring formed by the coupling element 2 and whose thickness D extends in a radial direction of the partial ring. In the exemplary embodiment, the cross-section of the strip 4 forming the coupling element 2 is constant over the entire circumference.

The coupling element 2 is partially cylindrical, it has the shape of a hollow cylinder which—in the exemplary embodiment-extends over approximately 270° in the circumferential direction and which—in the exemplary embodiment—has a circular or partially circular cross-section. An inner circumferential surface 6 of the coupling element 2, with which the coupling element 2 rests against the auditory ossicle when the coupling element 2 is placed on the auditory ossicle, is partially cylindrical.

The coupling element 2 has supports 7 projecting laterally, i.e. parallel to the axis, on both of its end faces 13. In the first embodiment of the invention shown in FIGS. 1 to 4, the supports 7 have the form of circular perforated discs which are short webs 8 are connected to the coupling element 2. In the exemplary embodiment, the coupling element 2, the webs 8 and the supports 7 are integral with one another.

In the exemplary embodiment, the supports 7 and the webs 8 are of the same thickness as the strip 4 of the coupling element 2 and, as can be seen in FIG. 3, they are arranged in alignment with the coupling element 2. The supports 7 are located in the same imaginary hollow cylinder as the coupling element 2 or as the strip 4 of the coupling element 2. In particular, inner surfaces 9 of the supports 7 are located on the same imaginary partial cylinder as the inner circumferential surface 6 of the coupling element 2 or as the inner circumferential surface 7 of the strip 4 of the coupling element 2. The inner surfaces 9 of the supports 7 rest against the auditory ossicle when the coupling element 2 is placed on the auditory ossicle.

The supports 7 can be regarded as components of the coupling element 2 or as elements of the auditory ossicular prosthesis 1 according to the invention protruding from the end faces 13 of the coupling element 2. In the circumferential direction, the supports 7 are short in relation to the coupling element 2. As can be seen in FIG. 4, the supports 7 extend radially onto the coupling element 2 over approximately half a diameter of the coupling element 2 in the circumferential direction. Because the supports 7 are connected to the coupling element 2 or the strip 4 of the coupling element 2 via the narrow webs 8, the supports 7 almost do not increase the flexural rigidity of the coupling element 2 in the radial direction of the coupling element 2.

The coupling element 2 can be located in a radial plane to its axis, i.e. its two end edges are located in two parallel planes. In the exemplary embodiment, the coupling element 2, as can be seen in FIGS. 2 and 4, is not arranged in a plane but is somewhat helical.

In the illustrated embodiment of the invention, the strip 4 of the coupling element 2 has slots 10 arranged obliquely to the circumferential direction and parallel to each other, which pass radially through the strip 4 and which are also regarded as elongated holes can. The slots 10 facilitate bending of the coupling element 2 to mold it to the auditory ossicle.

In the illustrated and described embodiment of the invention, the prosthesis body 3 is straight and rod-shaped and can also be understood as the shaft of the auditory ossicle prosthesis 1 according to the invention. The prosthesis body 3 is—in the exemplary embodiment—arranged at one end of the coupling element 2 and protrudes—in the exemplary embodiment radially—outwards from the coupling element 2.

In the embodiment of the invention shown in FIGS. 1 to 4, the supports 7 are arranged opposite the prosthesis body 3 on the coupling element 2, i.e. the two supports 7 are located in a plane with the prosthesis body 3, this common plane being an axial plane of the coupling element 2. The supports 7 support the coupling element 2 and thus also the auditory ossicle prosthesis 1 according to the invention against tilting about a radial axis of the coupling element 2, which lies in a common plane of the coupling element 2 and the prosthesis body 3, on the auditory ossicle when the coupling element 2 is placed on the auditory ossicle in such a way that the coupling element 2 encloses the auditory ossicle and rests with its inner circumferential surface 6 on the auditory ossicle and the supports 7 on both sides of the coupling element 2 rest on the auditory ossicle.

For a mechanical fastening of the auditory ossicle prosthesis 1 according to the invention in the human ear, the prosthesis body 3 is inserted into or through a hole in a footplate of a stirrup (not shown), which has previously been drilled through the footplate, such that an end of the prosthesis body 3 remote from the coupling element 2 projects into the inner ear. Instead, it is also possible, for example, to attach it to an oval window or to the stirrup with a second coupling element (not shown), which is or will be arranged at a distance from the coupling element 2 on the prosthesis body 3.

The coupling element 2 is snapped onto another auditory ossicle (not shown) in such a way that the coupling element 2 encloses the auditory ossicle. In particular, the coupling element 2 is snapped onto a rod-shaped section of the auditory ossicle, for example onto a short or a long leg of an anvil.

Holes 11 in the supports 7 allow a drop of water to be introduced between the supports 7 and the auditory ossicle. The water is distributed between the inner surfaces 9 of the supports 7 and the auditory ossicle and improves the hold of the supports 7 on the auditory ossicle through adhesion. In addition, the holes 11 in the supports 7 extend edges of the supports 7, which increases the adhesion.

The two supports 7, which support the auditory ossicles 1 against tilting on the auditory ossicle, facilitate an alignment of the auditory ossicle prosthesis 1 in the human ear and assist a surgeon in the implantation of the auditory ossicle prosthesis 1 according to the invention into the human ear. The alignment of the ossicular prosthesis 1 in the human ear is essential for good sound transmission from a tympanic membrane to the oval window or into the inner ear.

A drop of water or other liquid introduced between the coupling element 2 and the supports 7 on the one hand and the auditory ossicle on the other hand increases the hold of the coupling element 2 on the auditory ossicle against tipping during implantation by adhesion and facilitates the surgeon's work.

In the auditory ossicle prostheses 1 according to the invention shown in FIGS. 5 to 10, the coupling element 2 or at least the strip 4 of the coupling element 2 and the prosthesis body 3 correspond to FIGS. 1 to 4, so that in this respect, for the explanation of FIGS. 5 to 10, reference can be made to the above explanations of FIGS. 1 to 4. Only the shape and/or arrangement of the supports 7 have been changed. All auditory ossicular prostheses 1 have in common that they have supports 7 projecting laterally from their coupling elements 2, and that the inner surfaces 9 of the supports 7, i.e. the inner sides of the supports 7, are located on the same partial cylindrical surface as the inner circumferential surface 6 of the coupling element 2 or the strip 4 of the coupling element 2. This means that the inner surfaces 9 of the supports 7 are aligned or are flush with the inner peripheral surface 6 of the coupling element 2 or the strip 4 of the coupling element 2.

In the embodiment of the invention shown in FIG. 5, the supports 7 are triangular when viewed radially onto the coupling element 2, have a hole 11 and are connected in one piece to the coupling element 2 or to the strip 4 of the coupling element 2 via a short web 8.

FIG. 6 shows a variant of an auditory ossicular prosthesis 1 according to the invention, the supports 7 of which do not have holes 11. Otherwise, the auditory ossicular prostheses 1 from FIGS. 5 and 6 are identical.

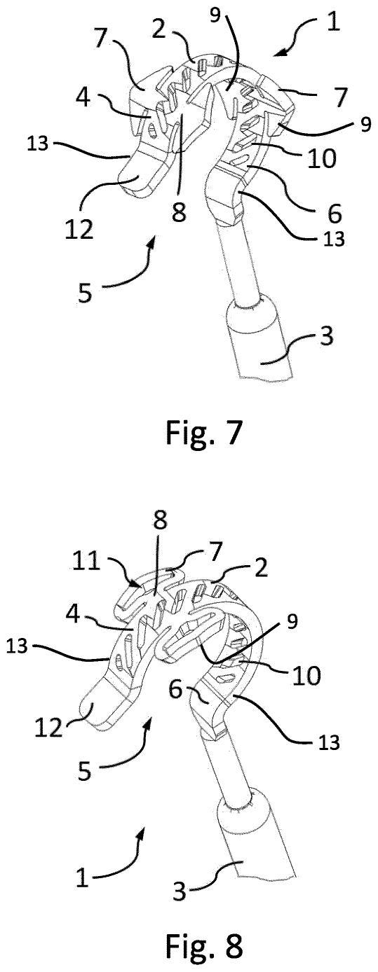

The auditory ossicle prosthesis 1 according to the invention shown in FIG. 7 has two pairs of supports 7, wherein two supports 7 of a pair are arranged opposite each other on the coupling element 2 and protrude in opposite directions laterally from the coupling element 2 or from the strip 4 of the coupling element 2. The two pairs of supports 7 are arranged offset on the coupling element 2 in the circumferential direction of the coupling element 2. Unlike the auditory ossicular prostheses 1 of FIGS. 1 to 6, the supports 7 of the auditory ossicular prosthesis 1 of FIG. 7 are located outside the axial plane of the coupling element 2 in which the prosthesis body 3 is located. This means that in FIG. 7 the supports 7 are not arranged opposite the prosthesis body 3 on the coupling element 2 and also not at the end 12 of the coupling element 2 from which the prosthesis body 3 protrudes. The supports 7 can, for example, be offset from one another by 180° in the circumferential direction of the coupling element 2; in FIG. 7, the supports 7 have a smaller offset from one another than 180° in the circumferential direction of the coupling element 2.

In the auditory ossicle prosthesis 1 according to the invention shown in FIG. 8, the supports 7 are almost oval and the supports 7 are arranged parallel to the circumferential direction of the coupling element 2 next to the strip 4 of the coupling element 2. In the exemplary embodiment, the supports 7 are almost oval rings, i.e. they have holes 11, and the supports 7 are integral with the coupling element 2 by means of short webs 8 or connected to the strip 4 of the coupling element 2. Supports 7 without the holes 11 (not shown) are also possible.

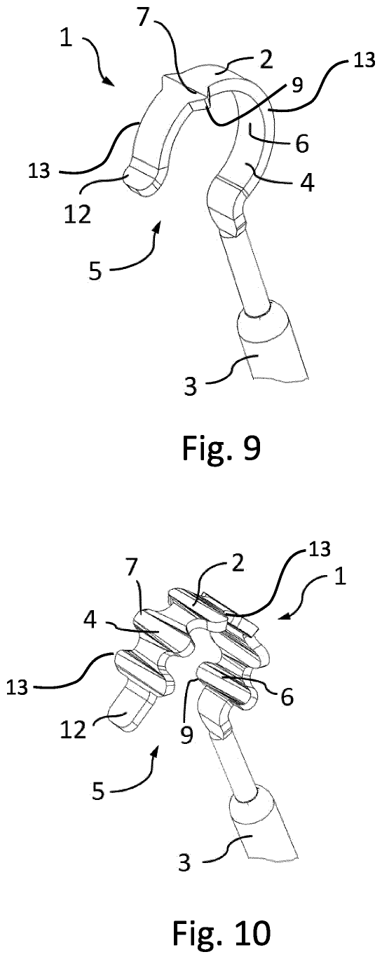

In the embodiment of the invention shown in FIG. 9, the supports 7 protrude laterally from the coupling element 2 or from the strip 4 of the coupling element 2 as triangular projections without webs. In addition, the coupling element 2 in FIG. 9 does not have any slots 10, which is also possible with the other embodiments of auditory ossicle prostheses 1 according to the invention.

In the auditory ossicle prosthesis 2 according to the invention shown in FIG. 10, the front edges of the coupling element 2 are wave-shaped, i.e. the strip 4 of the coupling element 2 widens and narrows alternately. The laterally outward-projecting waves of the end edges of the coupling element 2 form a plurality of supports 7 projecting laterally from the coupling element 2 on both sides. Instead of being wave-shaped, the supports 7 or the end edges of the coupling element 2 can also be triangular, sawtooth-shaped, rectangular, trapezoidal and so on (not shown).

1. An auditory ossicle prosthesis, comprising:
a partially ring-shaped coupling element with two lateral end faces configured to attach to an auditory ossicle, wherein the coupling element and the end faces extend over more than 180° and less than 360° in a circumferential direction and have an opening configured to place the coupling element on the auditory ossicle, wherein the coupling element has at least one support projecting laterally from one of the two end faces in the circumferential direction, the support supporting the coupling element on the auditory ossicle against tipping when the coupling element is placed on the auditory ossicle so-as-to enclose the auditory ossicle.

2. Auditory ossicle prosthesis according to claim 1, wherein the coupling element has a cylinder-shell-shaped or conical-shell-shaped inner circumferential surface of an imaginary cylinder or cone which the coupling element encloses, and an inner surface of the support projects on a circumferential surface laterally to the circumferential direction in extension of the imaginary cylinder or cone.

3. Auditory ossicle prosthesis according to claim 1, wherein the coupling element has supports on both end faces.

4. Auditory ossicle prosthesis according to claim 1, wherein the coupling element has a plurality of supports which are offset from one another in the circumferential direction of the coupling element.

5. Auditory ossicle prosthesis according to claim 4, wherein the coupling element has no more than five, four, three or two supports on one end face.

6. Auditory ossicle prosthesis according to claim 1, wherein the coupling element has a wave-shaped or toothed front edge, wherein waves or teeth of the front edge form the supports.

7. Auditory ossicle prosthesis according to claim 1, wherein the support is shorter in the circumferential direction than 10 percent of a length of the end face.

8. Auditory ossicle prosthesis according to claim 1, wherein the coupling element has a partially annular strip which has a constant width (B) in the circumferential direction outside the support(s).

9. Auditory ossicle prosthesis according to claim 1 wherein the coupling element is helical.

10. Auditory ossicle prosthesis according to claim 1, wherein the support has a hole in the radial direction or a perforated disc or is polygonal.

11. An auditory ossicle prosthesis according to claim 1, wherein the auditory ossicle prosthesis has a prosthesis body projecting outwardly from the coupling element and the support is arranged laterally on the coupling element in extension of the prosthesis body.

12. Auditory ossicle prosthesis according to claim 1, wherein the coupling element has elongated holes or slots which are arranged obliquely to the circumferential direction.
